(19) United States
(12) Patent Application Publication
(10) Pub. No.: US 2006/0025446 A1
(43) Pub. Date: Feb. 2, 2006

(54) Propargyl nitroxides and indanyl nitroxides and their use for the treatment of neurologic diseases and disorders

(76) Inventors: Jeffrey Sterling, Jerusalem (IL); Benjamin Sklarz, Petach-Tikva (IL); Yaacov Herzig, Raanana (IL); David Lerner, Jerusalem (IL); Eliezer Falb, Givataim (IL); Haim Ovadia, Jerusalem (IL)

Correspondence Address: COOPER & DUNHAM, LLP, 1185 AVENUE OF THE AMERICAS, NEW YORK, NY, 10036, US

(21) Appl. No.: 11/191,212

(22) Filed: Jul. 26, 2005

Related U.S. Application Data
(60) Provisional application No. 60/591,819, filed on Jul. 27, 2004.

(51) Int. Cl. C07D 211/06; A61K 31/445

(52) U.S. Cl. 514/319; 546/206

(57) ABSTRACT

Disclosed are compounds having the structure: 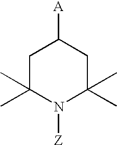 wherein Z is —OH or —O•; and
A is: 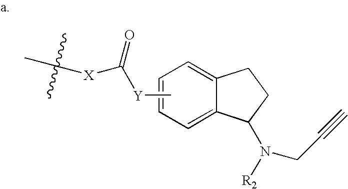 wherein
X and Y are independently NR₁ or O, where
R₁ is H or C₁-C₄ alkyl; and
R₂ is H, C₁-C₄ alkyl or t-butoxycarbonyl, 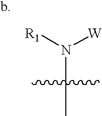
wherein W is C₃-C₄ alkynyl; and
R₁ is H or C₁-C₄ alkyl, or 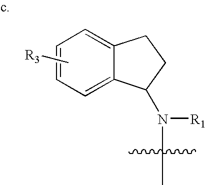
wherein R₁ is H, C₁-C₄ alkyl, or C₃-C₄ alkynyl; and
   R₃ is H, OH, O(C₁-C₄ alkyl), or a halogen,
      optically active enantiomers, pharmaceutically acceptable salts of the compounds, pharmaceutical compositions containing such compounds or salts, and processes for their preparation. The subject invention also provides methods of alleviating symptoms of neurologic, autoimmune, and inflammatory disorders caused by the presence of reactive oxygen species, methods of preventing oxidation of lipids, proteins, or deoxyribonucleic acids on a cellular level, and methods of protecting human red blood cells from lysis by O₂ radicals.

This application claims benefit of U.S. Provisional Application No. 60/591,819, filed Jul. 27, 2004, the contents of which are hereby incorporated by reference.

Throughout this application various publications are referenced in parenthesis. The disclosures of these publications in their entireties are hereby incorporated by reference into this application in order to more fully describe the state of the art to which this invention pertains.

### BACKGROUND OF THE INVENTION

Neurologic diseases and disorders are becoming increasingly common in North America. For example, Parkinson's disease is a common neurologic disorder, affecting nearly 1 million people in North America. Thus, developing an effective treatment for neurologic disorders has become a high priority in the drug industry.

Neurologic diseases can generally be divided into two groups based on their physiological and pathological characteristics. Parkinson's disease, Alzheimer's disease, Huntington's disease and amyotrophic lateral sclerosis (ALS or Lou Gehrig's disease) are all progressive disorders (i.e., their symptoms are not apparent until months or more commonly years after the disease has begun), caused by an initial reduction of neuronal function, followed by a complete loss of function upon neuronal death. In addition, these progressive neurologic disorders are characterized by the presence of protein aggregates that are believed to hamper cellular functions (e.g., neurotransmission) and may ultimately result in cell death (Sasaki et al. Am. J. Pathol., (1998) 153:1149-55).

Multiple sclerosis is a disease of the central nervous system, which is slowly progressive and is characterized by disseminated patches of demyelination in the brain and spinal cord, resulting in multiple and varied neurological symptoms and signs, usually with remissions and exacerbations. The cause is unknown but an immunologic abnormality is suspected (THE MERCK MANUAL, 17th EDITION, 1999 MERCK & CO.) Several different drug therapies are currently being investigated.

While the aforementioned diseases are all slowly progressive, neurological dysfunction can also be caused by a more abrupt event such as an infarction of brain tissue, or stroke. Brain stroke is the third leading cause of death in developed countries. Survivors often suffer from neurological and motor disabilities. The majority of central nervous system (“CNS”) strokes are regarded as localized tissue anemia following obstruction of arterial blood flow which causes oxygen and glucose deprivation. R(+)-N-propargyl-1-aminoindan has been shown to be an effective treatment for stroke and traumatic brain injury (U.S. Pat. No. 5,744,500).

A series of propargylamines, including Selegiline and Rasagiline, have been shown to prevent apoptosis in dopamine neurons in Parkinson's models (Naoi, M. et al. J. Neural Transmission (2002) 109:607-721). R(+)N-propargyl-1-aminoindan has recently been suggested as being useful for treating Parkinson's disease, dementia and depression (U.S. Pat. No. 5,453,446). The neuroprotective activity of these molecules is attributed by some to the presence of the propargyl moiety (Youdim, M. B. H. et al. Biochem. Pharmacol. (2003) 66:1635-41). The mechanism by which the propargyl moiety may confer neuroprotection is not fully understood. However, it is clear that the mechanism involves a complex set of neurochemical events including alterations in Bcl-2, GAPDH, SOD and catalase (Youdim, M. B. H. Cell. Mol. Neurobiol. (2001) 21(6): 555-73).

Nitroxides, which are cell-permeable, nontoxic, non-immunogenic stable radicals, have been used as biophysical probes for monitoring membrane stability, cellular pH, oxygen concentration, intracellular redox reactions, and as contrast agents for MRI (Shohami, E. et al. J. Cerebral Blood Flow Metab. (1997) 17:1007-19). These compounds undergo a one electron redox reaction and catalyze the dismutation of oxygen radicals. They can also reduce hypervalent metals and catalytically facilitate H₂O₂ transformations by hemeproteins (Shohami, E. et al. J. Cerebral Blood Flow Metab. (1997) 17:1007-19). They have been shown to prevent ROS (reactive oxygen species)-mediated lipid peroxidation and to selectively detoxify paramagnetic species including radicals and transition metals. By undergoing one-electron-transfer reactions, nitroxides are readily reduced to hydroxylamines or oxidized to the oxoammonium cation. Nitroxides have been shown to be metabolized in biological systems to the corresponding hydroxylamines through enzyme-catalyzed mechanisms (Offer, T. and Samuni, A. Free Radical Biol. Med. (2002) 32, 872-81; Zhang, R. et al. Free Radical Biol. Med., (1998) 24, 332-40). Piperidine nitroxides, such as TEMPO (2,2,6,6-tetramethylpiperidine-1-nitroxide); TEMPOL (4-hydroxy-2,2,6,6-tetramethylpiperidine-1-nitroxide,); and Tempamine (4-amino-2,2,6,6-tetramethylpiperidine-1-nitroxide) were shown to be efficient antioxidants in the rat CHI (closed head injury) model (Shohami, E. et al. J. Cerebral Blood Flow Metab. (1997) 17:1007-19). These compounds were effective in protecting brain tissue by terminating radical chain reactions, by oxidizing deleterious metal ions and by removing intracellular superoxide (Ibid.) It has also been suggested that the observed protecting effect of such nitroxides may be attributed to oxidation of Fe²⁺ and subsequent blocking of iron-dependent processes crucial for production of damage-inducing oxidants (Glebska, J. et al. Biometals (2001) 14:159-70).

Piperidine nitroxides and their derivatives were thoroughly investigated by, inter alia, Hideg and Krishna (Krishna, M. C. et al. J. Med. Chem. (1998) 41:3477-92). These authors have shown that protection against H₂O₂-induced toxicity was influenced mainly by ring size, redox-potential and oxidation state. Radioprotection was found to be determined by ring substitution and oxidation state. No cytotoxicity was observed for the compounds screened in this work.

The reduced (hydroxylamine) form of TEMPOL (4-hydroxy-2,2,6,6-tetramethylpiperidine-1-hydroxypiperidine) was reported to protect cardiomyocytes from oxidative stress in a manner comparable to TEMPOL (Zhang, R. et al. Free Radical Biol. Med. (1998) 24:66-75).

ROS have been shown to be involved in demyelination and in demyelinating diseases, including multiple sclerosis (Smith K. J. et al. Brain Pathol. (1999) 9:69-92). In particular, peroxynitrite and the highly reactive hydroxyl radical oxidize DNA, proteins and initiate lipid peroxidation which in turn may lead to demyelination and neuronal damage (Karg, E. et al. J. Neurol. (1999) 246:533-39). Free radicals may also contribute to the damage of the blood-brain-barrier, which is an early event of multiple sclerosis lesions (Frank, J. A. et al. Ann. Neurol. (1994) 36[Suppl]:S86-S90). An imidazoline nitroxide (compound 2) has been reported to effectively prevent the development of EAE in an acute model (Hooper, D. C. et al. Proc. Natl. Acad. Sci. U.S.A. (1997) 94:2528-33). EUK-8, a synthetic catalytic scavenger of ROS has also been reported to prevent and suppress EAE (Malfroy, B. et al. Cell. Immunol. (1997) 177:62-8).

Compound 1a was reported as a toxic, stable nitroxyl radical (potentially useful as an anti-cancer agent). Compound 1b was reported as a side product obtained in the preparation of spin-labeled adenosine derivatives (Anzai, B. et al, J. Org. Chem., (1982) 47, 4281-5).

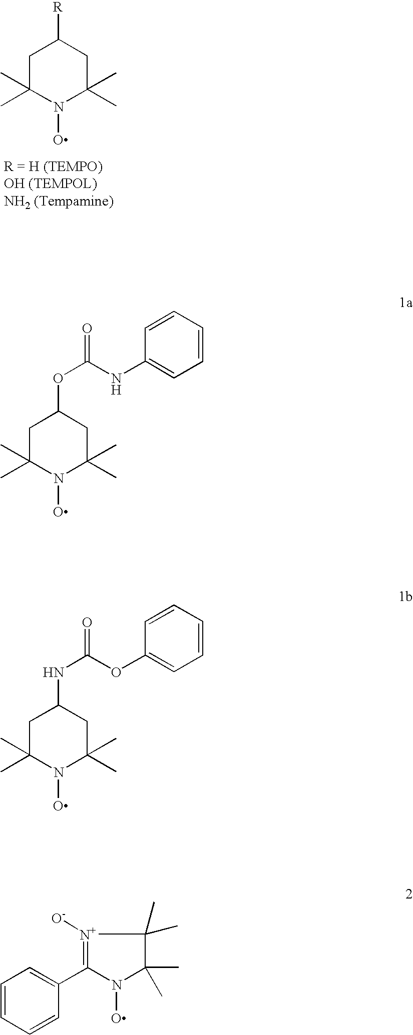

### SUMMARY OF THE INVENTION

The subject invention provides a compound having the structure: 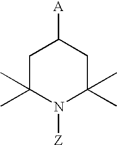
wherein Z is —OH or —O•; and
A is: 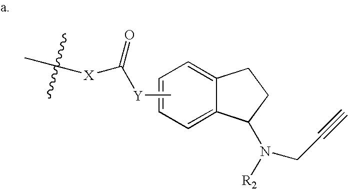
   wherein
   X and Y are independently NR₁ or O, where
   R₁ is H or C₁-C₄ alkyl; and
   R₂ is H, C₁-C₄ alkyl or t-butoxycarbonyl, 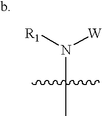
   wherein W is C₃-C₄ alkynyl; and
   R₁ is H or C₁-C₄ alkyl, or 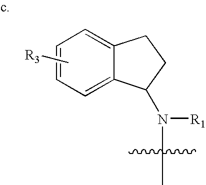
   wherein R₁ is H, C₁-C₄ alkyl, or C₃-C₄ alkynyl; and
   R₃ is H, OH, O(C₁-C₄ alkyl), or a halogen,
or an enantiomer or a pharmaceutically acceptable salt thereof.

The subject invention also provides a process of manufacturing a compound having the structure: 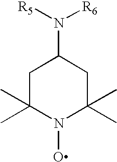
wherein R₅ is C₃-C₄ alkynyl or an indan-1-yl group and R₆ is H or C₁-C₄ alkyl, comprising:
   a. reacting a compound having the structure: 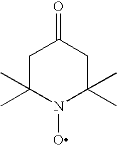
with a compound having the structure: 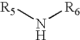
wherein R₅ and R₆ are defined as above, in the presence of a reducing agent to form the compound.

### DESCRIPTION OF THE FIGURES

FIG. 1: General synthesis scheme for production of the disclosed compounds.

FIG. 2: Synthesis of compounds A10 and A11.

FIG. 3: Synthesis of compounds B10, B11, B12, and C10.

FIGS. 4 and 5: Neuroprotective activity of compound A11 (MPP+).

FIG. 6: Percent neuroprotection of compound A11 at various concentrations.

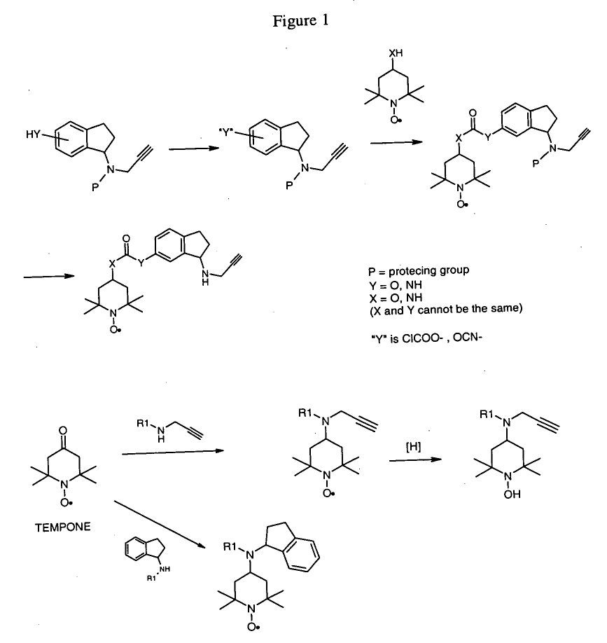

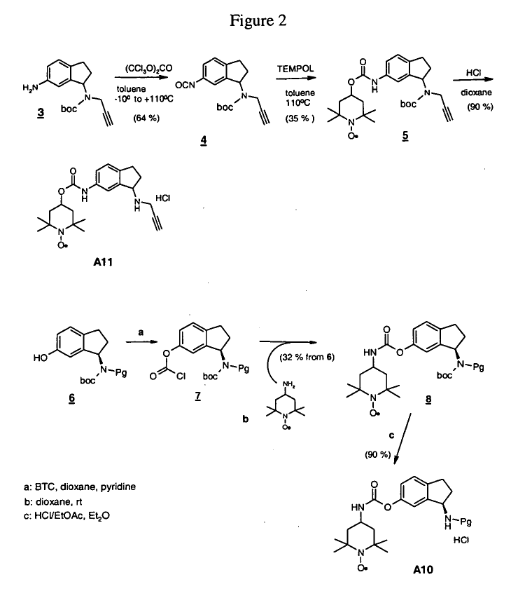

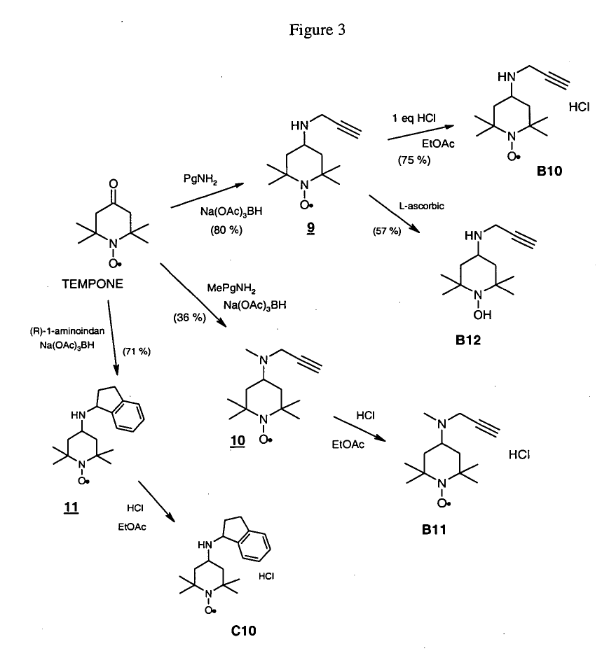

### DETAILED DESCRIPTION OF THE INVENTION

The subject invention provides a compound having the structure: 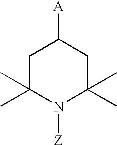
wherein Z is —OH or —O•; and
A is: 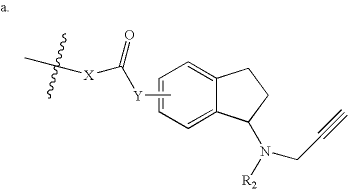
   wherein
   X and Y are independently NR₁ or O, where
   R₁ is H or C₁-C₄ alkyl; and
   R₂ is H, C₁-C₄ alkyl or t-butoxycarbonyl, 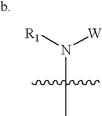
wherein W is C₃-C₄ alkynyl; and
   R₁ is H or C₁-C₄ alkyl, or 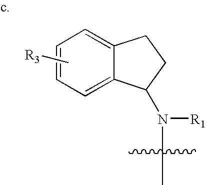
   wherein R₁ is H, C₁-C₄ alkyl, or C₃-C₄ alkynyl;
   and R₃ is H, OH, O(C₁-C₄ alkyl), or a halogen,
or an enantiomer or a pharmaceutically acceptable salt thereof.

In a further embodiment, the compound has the structure: 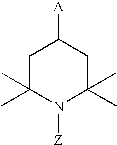
wherein Z is —OH or —O•; and
A is: 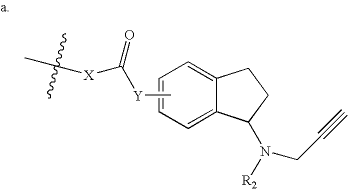
   wherein
   X and Y are independently NR₁ or O, where
   R₁ is H or C₁-C₄ alkyl; and
   R₂ is H, C₁-C₄ alkyl, 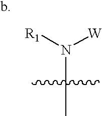
   wherein W is C₃-C₄ alkynyl; and
      R₁ is H or C₁-C₄ alkyl, or 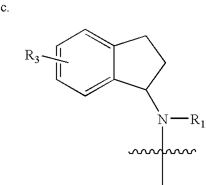
   wherein R₁ is H, C₁-C₄ alkyl, or C₃-C₄ alkynyl;
   and R₃ is H, OH, O(C₁-C₄ alkyl), or a halogen, or an enantiomer or a pharmaceutically acceptable salt thereof.

In a further embodiment, A is: 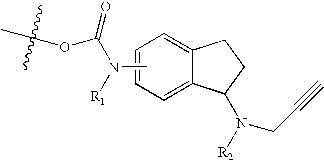
wherein R₁ is H or C₁-C₄ alkyl; and R₂ is H or C₁-C₄ alkyl, or an enantiomer or a pharmaceutically acceptable salt thereof.

In a further embodiment, R₁ and R₂ are H. In a further embodiment, Z is —O•. In a further embodiment, the compound is (3-prop-2-ynylamino-indan-5-yl) carbamic acid 2,2,6,6-tetramethyl-1-piperidinyloxy-4-yl ester HCl.

In a further embodiment A is: 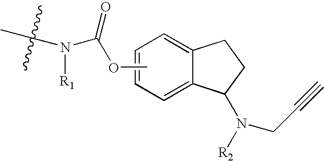
wherein R₁ is H or C₁-C₄ alkyl; and R₂ is H or C₁-C₄ alkyl, or an enantiomer or a pharmaceutically acceptable salt thereof. In a further embodiment, R₁ and R₂ are H. In a further embodiment, Z is —O•. In a further embodiment, the compound is (2,2,6,6-tetramethyl-1-piperidinyloxy-4-yl)-carbamic acid 3-(R)-prop-2-ynylamino-indan-5-yl ester HCl.
   In a further embodiment A is: 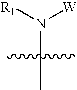
wherein W is C₃-C₄ alkynyl; and R₁ is H or C₁-C₄ alkyl, or a an enantiomer or pharmaceutically acceptable salt thereof.

In a further embodiment, A is:

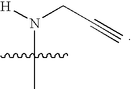

In a further embodiment, Z is —O•. In a further embodiment, the compound is 2,2,6,6-tetramethyl-4-prop-2-ynylamino-1-piperidine nitroxide HCl. In a further embodiment, the compound is 2,2,6,6-tetramethyl-4-prop-2-ynylamino-piperidin-1-ol.

In a further embodiment, Z is —OH. In a further embodiment, the compound is 2,2,6,6-tetramethyl-4-prop-2-ynylamino-piperidin-1-ol 2 HCl.

In a further embodiment, A is:

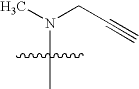

In a further embodiment, Z is —O•. In a further embodiment, the compound is 2,2,6,6-tetramethyl-4-(methyl-prop-2-ynylamino)-1-piperidine nitroxide HCl.

In a further embodiment, A is 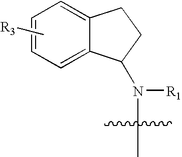
wherein R₁ is H, C₁-C₄ alkyl, or C₃-C₄ alkynyl; and R₃ is H, OH, O(C₁-C₄ alkyl), or a halogen, or an enantiomer or a pharmaceutically acceptable salt thereof.

In a further embodiment, R₁ and R₃ are H. In a further embodiment, Z is —O•. In a further embodiment, the compound is 4-(indan-1-ylamino)-2,2,6,6-tetramethyl-1-piperidine nitroxide HCl.

In a further embodiment, the compound is an optically active enantiomer.

In a further embodiment, the compound has the structure: 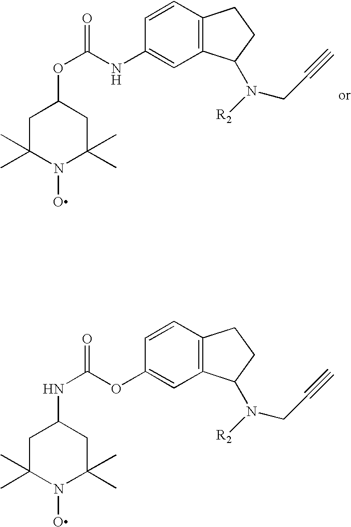
wherein R₂ is a t-butoxycarbonyl group.

The subject invention also provides a process of manufacturing the compound comprising:
a. reacting 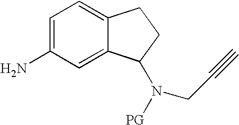
   wherein PG is a protecting group, with (CCl₃O)₂CO to form: 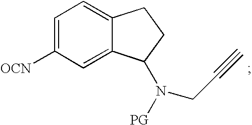
b. reacting the product of a. with 4-hydroxy-2,2,6,6-tetramethylpiperidine-1-nitroxide to form: 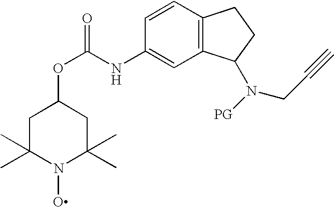
   wherein PG is a protecting group; and
c. reacting the product of step b. with an acid to form:

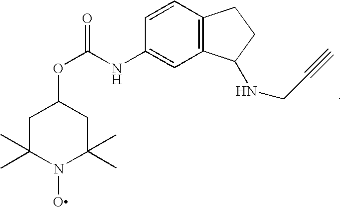

In an embodiment, PG is a t-butoxycarbonyl group. In a further embodiment, the acid is HCl.

In a further embodiment, the process comprises:
a. reacting 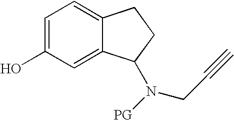
   wherein PG is a protecting group, with bis-(trichloromethyl) carbonate to form: 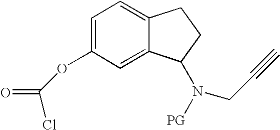
b. reacting the product of step a. with 4-amino-2,2,6,6-tetramethyl piperidine-1-nitroxide to form: 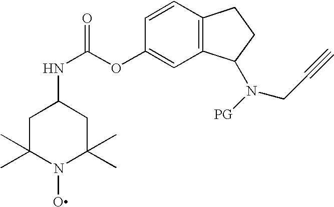
   wherein PG is a protecting group; and
c. reacting the product of step b. with an acid to form:

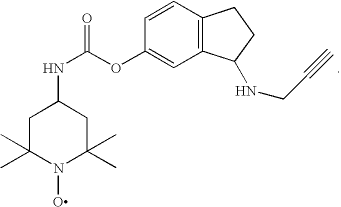

In an embodiment, PG is a t-butoxycarbonyl group.

The subject invention also provides a process of manufacturing a compound having the structure: 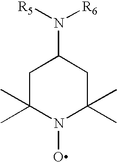
wherein R₅ is C₃-C₄ alkynyl or an indan-1-yl group and R₆ is H or C₁-C₄ alkyl, comprising:
   reacting a compound having the structure: 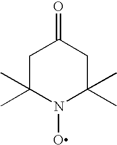
   with a compound having the structure: 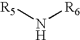
   wherein R₅ and R₆ are defined as above, in the presence of a reducing agent to form the compound.

In an embodiment, R₅ is a propargyl group, R₆ is H and the reducing agent is sodium triacetoxyborohydride. In a further embodiment, R₅ is a propargyl group, R₆ is CH₃ and the reducing agent is sodium triacetoxyborohydride. In a further embodiment, R₅ is an indan-1-yl group and R₆ is H and the reducing agent is sodium triacetoxyborohydride.

The subject invention also provides a method of treating a subject suffering from a neurologic disorder or an autoimmune disorder, comprising administering to the subject a therapeutically effective amount of any one of the compounds disclosed herein so as to thereby treat the subject. In one embodiment, the subject suffers from a neurologic disorder. In a further embodiment, the neurologic disorder is Alzheimer's disease. In a further embodiment, the neurologic disorder is Parkinson's disease. In a further embodiment, the neurologic disorder is amyotrophic lateral sclerosis.

In a further embodiment, the subject suffers from an autoimmune disorder. In a further embodiment, the autoimmune disorder is multiple sclerosis.

The subject invention also provides a method of treating a subject afflicted with an inflammatory disorder caused by the presence of reactive oxygen species, comprising administering to the subject a therapeutically effective amount of any one of the compounds disclosed herein so as to thereby treat the subject.

In an embodiment, the inflammatory disorder is an autoimmune inflammatory disorder. In a further embodiment, the autoimmune inflammatory disorder is caused by the presence of peroxynitrite in the subject. In a further embodiment, the autoimmune inflammatory disorder is an inflammatory bowel disease. In a further embodiment, the autoimmune inflammatory disease is rheumatoid arthritis.

The subject invention also provides a method of preventing the oxidation of lipids, proteins, or deoxyribonucleic acid in a cell, comprising contacting the cell with any one of the compounds disclosed herein.

The subject invention also provides a method of preventing lysis of human red blood cells by oxygen radicals, comprising contacting the cells with any one of the compounds disclosed herein.

The subject invention also provides a pharmaceutical composition comprising any one of the compounds disclosed herein and a pharmaceutically acceptable carrier.

The subject invention also provides a process for the manufacture of a pharmaceutical composition comprising admixing any one of the compounds disclosed herein with a pharmaceutically acceptable carrier.

The subject invention also provides a packaged pharmaceutical composition for treating Alzheimer's disease, Parkinson's disease, multiple sclerosis, or an autoimmune inflammatory disorder which is caused by the presence of reactive oxygen species in a subject comprising: a. a pharmaceutical composition of the subject invention; and b. instructions for using the composition for treating Alzheimer's disease, Parkinson's disease, multiple sclerosis, amyotrophic lateral sclerosis, or the inflammation which is caused by the presence of reactive oxygen species in the subject.

In an embodiment, the pharmaceutical composition of the instant invention is for use in treating a neurologic disorder, an autoimmune disorder, an inflammatory disorder, or an autoimmune inflammatory disorder in a subject.

The subject invention also provides for the use of any one of the compounds disclosed herein for manufacturing a medicament useful for treating a subject suffering from a neurologic disorder or an autoimmune disorder.

In an embodiment, the subject suffers from a neurologic disorder. In a further embodiment, the neurologic disorder is Alzheimer's disease. In a further embodiment, the neurologic disorder is Parkinson's disease. In a further embodiment, the neurologic disorder is amyotrophic lateral sclerosis.

In a further embodiment, the subject suffers from an autoimmune disorder. In a further embodiment, the autoimmune disorder is multiple sclerosis.

The subject invention also provides for the use any one of the compounds disclosed herein for manufacturing a medicament useful for treating a subject afflicted with an inflammatory disorder caused by the presence of reactive oxygen species.

In an embodiment, the inflammatory disorder is an autoimmune inflammatory disorder. In a further embodiment, the autoimmune inflammatory disorder is caused by the presence of peroxynitrite in the subject. In a further embodiment, the autoimmune inflammatory disorder is an inflammatory bowel disease. In a further embodiment, the autoimmune inflammatory disorder is rheumatoid arthritis.

The subject invention also provides for the use of any one of the compounds disclosed herein for manufacturing a medicament useful for preventing the oxidation of lipids, proteins or deoxyribonucleic acid in a cell.

The subject invention also provides the use of any one of the compounds disclosed herein for manufacturing a medicament useful for preventing lysis of human red blood cells by oxygen radicals.

The following table correlates the compound number, structure and IUPAC name of several disclosed subject compounds:

| Compound | | |
|---|---|---|
| Number | Structure | IUPAC name |
| B10 | 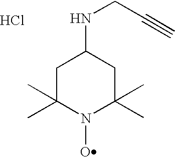 | 2,2,6,6-tetramethyl-4- propynylamino-1-piperidine nitroxide HCl |
| A10 | 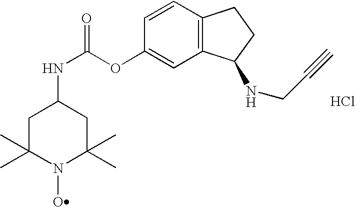 | (2,2,6,6-tetramethyl-1- piperidinyloxy-4-yl)- carbamic acid 3-(R)- prop-2-ynylamino-indan-5-yl ester HCl |
| B11 | 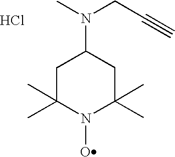 | 2,2,6,6-tetramethyl-4- (methyl-propynylamino)-1- piperidine nitroxide HCl |
| A11 | 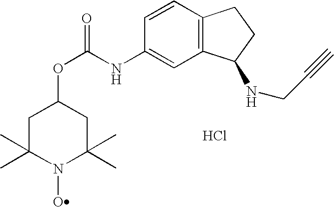 | (3-prop-2-ynylamino-indan-5-yl) carbamic acid 2,2,6,6-tetramethyl-1- piperidinyloxy-4-yl ester HCl |
| C10 | 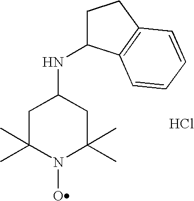 | 4-(indan-1-ylamino)- 2,2,6,6-tetramethyl-1- piperidine nitroxide HCl |
| B12 | 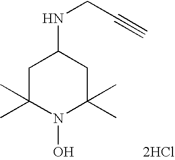 | 2,2,6,6-tetrarnethyl-4- prop-2-ynylamino- piperidin-1-ol 2HCl |

The present invention provides novel derivatives of propargyl nitroxides and indanyl nitroxides which, by virtue of comprising the two moieties, a nitroxide or hydroxylamine moiety and a propargylamine or indanyl moiety, are effective as neuroprotectants and for treatment of neurologic disorders, including multiple sclerosis.

It will be noted that the structure of some of the compounds of this invention includes asymmetric carbon atoms and thus occur as racemates and racemic mixtures, single enantiomers, diastereomeric mixtures and individual diastereomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis.

The disclosed compounds have the neuroprotective properties of propargylaminoindans (PAI's) and propargylamine (Pg) moieties, and the antioxidant/radical scavenging activity of the piperidine nitroxides. These compounds act as neuroprotectants and antioxidants for the treatment of neurologic diseases such as multiple sclerosis, Alzheimer's disease, and Parkinson's disease. In target compounds A10 and A11, the two moieties are linked via a carbamate moiety, and in compounds B10, B11, and B12, the propargylamine moiety is connected directly to the piperidine nitroxide moiety. Target compound C10 comprises an aminoindan functionality which confers additional lipophilicity and blood-brain barrier permeability.

An oxygen free radical is depicted herein as: —O•.

The phrase “neurologic disorder” as used herein refers to a disorder whose adverse affects are localized in the nervous system.

The phrase “autoimmune disorder” as used herein refers to a disorder in which the immune system produces autoantibodies to an endogenous antigen, with consequent injury to tissues.

The phrase “autoimmune inflammatory disorder” as used herein refers to a disorder in which the immune system triggers an inflammatory response without any foreign substances present.

The phrase “protecting group” as used herein refers to a removable chemical unit used in synthetic chemistry to intentionally block a region of a molecule so as to prevent that region from reacting during a given reaction.

The following abbreviations are used throughout the application:

| | |
|---|---|
| RT | Room temperature |
| Boc | t-butoxycarbonyl |
| EtOAc | Ethyl acetate |
| Et₂O | Ether |
| COSY | Correlated Spectroscopy |
| Mp | Melting point |

The invention is further illustrated by the following examples which in no way should be construed as being further limiting. The contents of all references, pending patent applications and published patent applications, cited throughout this application, including those referenced in the background section, are hereby incorporated by reference. It should be understood that the models used throughout the examples are accepted models and that the demonstration of efficacy in these models is predictive of efficacy in humans.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

Introduction—General Syntheses:

Propargylaminoindanyl TEMPO carbamates (Compounds A10 and A11) were prepared by reacting suitably N-protected (for example, protected by a t-butoxycarbonyl group) hydroxy or amino propargylaminoindans with bis(trichloromethyl) carbonate (BTC), followed by reacting either the isocyanato or the chloroformate derivatives with Tempol or Tempamine, respectively. Finally, the Boc protecting group was removed by acidolysis under anhydrous conditions, such as HCl in dioxane or EtOAc/Et₂O.

N-Propargyl tempamines (B10 and B11) were prepared by reductive alkylamination of tempone by reacting tempone with alkynylamines in the presence of a reducing agent such as sodium acetoxyborohydride in a suitable solvent such as dichloroethane, followed by converting the free bases to the corresponding hydrochlorides by HCl (1.1 molar excess) in ethyl acetate. Indanyl tempamine (C10) was also obtained by reductive alkylamination of tempone by reacting tempone with aminoindan as described above.

Propargyl hydroxylamines, such as B12, were obtained by reducing the propargyl nitroxides, by an agent such as ascorbic acid.

In a further embodiment of the invention, conditions were devised to selectively remove the N-Boc protecting group (a carbamate) in the presence of the carbamate linker functionality.

The structures of the target compounds were established based on ¹H (COSY, HMBC (heteronuclear multiple bond correlation)), ¹³C NMR and ESR spectra.

### Example 1

### N-Boc-(6-aminoindan-1-yl)-prop-2-ynylamine (Compound 3)

6-Nitroindanone (6.86 g, 38.72 mmol) was dissolved in 1,2 dichlorethane (220 mL), and a solution of propargylamine (2.68 g, 48.66 mmol) in dichloroethane (15 mL) was added. The mixture was stirred at 25° C. under nitrogen for 30 min and sodium triacetoxyborohydride (13.42 g, 63.32 mmol) was added neat. The mixture was then stirred at 25° C. under nitrogen for 50 h. Solvent was evaporated under reduced pressure to give a dark solid residue. The residue was treated with ethyl acetate (300 mL) and the mixture was stirred at 45° C. for 1 h and filtered. Silica gel was added to the filtrate and the mixture was evaporated to dryness under vacuum to give silica gel impregnated with the crude product. This was placed on top of a silica gel column and purified by flash column chromatography (hexane:ethyl acetate 25:75) to give 5.80 g (69%) of (6-Nitro-indan-1-yl)-prop-2-ynylamine as a brown solid, mp 37-39° C.

(6-Nitro-indan-1-yl)-prop-2-ynylamine (6.0 g, 27.74 mmol) was dissolved in absolute ethanol (130 mL) and a solution of di-t-butyl dicarbonate (6.24 g, 28.56 mmol) in absolute ethanol (30 mL) was added dropwise with stirring over 15 min. The solution was then stirred at 25° C. under nitrogen for 24 h. The solvent was evaporated to dryness under reduced pressure to give a dark viscous oil. Hexane (70 mL) was added to the viscous oil and the mixture was stirred for 20 min and the hexane was decanted off. This procedure (adding hexane, stirring for 20 min and decanting off the hexane) was repeated nine more times. The combined hexane washings were evaporated to dryness under reduced pressure to give 8.20 g (93%) of N-Boc-(6-nitro-indan-1-yl)-prop-2-ynylamine as a light tan solid, mp 56-59° C.

N-Boc-(6-nitro-indan-1-yl)-prop-2-ynylamine (5.55 g, 17.54 mmol) and stannous chloride dihydrate (19.76 g, 87.59 mmol) were dissolved in anhydrous ethanol (320 mL) and heated to 60° C. Sodium borohydride (1.33 g, 35.16 mmol) dissolved in ethanol (70 mL) was then added dropwise with stirring under nitrogen over 30 min. The stirred mixture was heated at 60° C. for 1.5 h, cooled to 10° C., diluted with cold water and the pH was adjusted to 7-8 by 25% NH₄OH, and EtOAc was added. The mixture was stirred for 10 min, filtered, water and brine were added, and the layers were separated; the aqueous layer was re-extracted with EtOAc. The combined organic layers were dried and evaporated to dryness under reduced pressure to give a crude viscous oil which was purified by flash column chromatography (hexane:ethyl acetate 50:50), to give 3.8 g (75%) of N-Boc-(6-aminoindan-1yl)-prop-2-ynylamine as a viscous yellow oil.

### Example 2

### (6-Isocyanato-indan-1-yl)-prop-2-ynyl-carbamic acid t-butyl ester (Compound 4)

A mixture of N-Boc-(6-aminoindan-lyl)-prop-2-ynylamine (Compound 3), (7.05 g, 24.62 mmol) and carbon black (850 mg) was suspended in dry toluene (200 mL) and cooled to −10° C. in an ice/salt water bath. A solution of triphosgene (3.63 g, 12.23 mmol) in dry toluene (50 mL) was then added dropwise with stirring over 20 min. The temperature was maintained at −5° C. during the addition. The mixture was allowed to warm up to 25° C. slowly, and it was then heated at reflux under nitrogen for 2.5 h, cooled to RT and filtered (filter-aid). Evaporation of the filtrate to dryness under reduced pressure gave 5.57 g (75.7% yield) of a viscous tan oil which was used without further purification.

### Example 3

### [6-(2,2,6,6-Tetramethyl-1-piperidinyloxy-4-yloxycarbonylamino)-indan-1-yl)]-prop-2-ynyl-carbamic acid t-butyl ester (Compound 5)

A solution of Tempol (3.07 g, 17.82 mmol) and (6-isocyanato-indan-1-yl)-prop-2-ynyl-carbamic acid t-butyl ester (Compound 4) (5.57 g, 17.83 mmol) in dry toluene (200 mL) was stirred and heated at reflux under nitrogen for 11 h. The dark solution was cooled to 25° C., silica gel (5.5 g) was added and the toluene was evaporated to dryness at reduced pressure. The impregnated silica gel was placed on top of a silica column and purified by flash column chromatography (hexane:ethyl acetate 70:30) to give 2.16 g (25% yield) of an orange solid.

### Example 4

### (3-Prop-2-ynylamino-indan-5-yl) carbamic acid 2,2,6,6-tetramethyl-1-piperidinyloxy-4-yl ester HCl (Compound A11)

Compound 5 (1.70 g, 3.51 mmol) was dissolved in dioxane (130 mL) and a 20% w/w solution of HCl gas in dioxane (60 mL) was added. The reaction mixture was stirred at 25° C. under nitrogen for 48 h, and evaporated to dryness to give a light-colored solid. Water (150 mL) and methylene chloride (150 mL) were added to the solid and the layers were separated. The methylene chloride layer was discarded. The aqueous layer was washed with CH₂Cl₂ (3×70 ml) and the aqueous layer was then carefully evaporated to dryness under vacuum to give a light tan solid. The solid was stirred first with warm hexane (80 mL) and the hexane was decanted. The solid was then stirred with anhydrous ether (100 mL) and the ether was decanted. The resulting solid was then dried under vacuum at 55° C. for 64 h to give 1.52 g (quantitative yield) of an off white solid.

MS: 386 (MH₂⁺, 14), 370 (7), 331 (14), 315 (8).

¹H NMR(δ, DMSO-d₆):

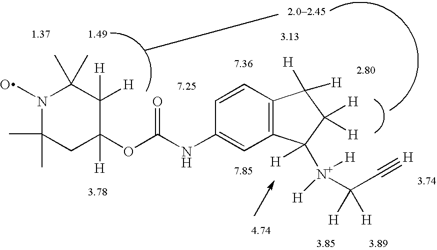

### Example 5

### 2,2,6,6-Tetramethyl-4-(prop-2-ynylamino)-piperidine nitroxide HCl (Compound B10)

Tempone (1.50 g, 8.81 mmol) was dissolved in 1,2-dichloroethane (DCE, 50 ml) and a solution of propargylamine (0.61 g, 11.07 mmol) in DCE (10 mL) was added. The mixture was stirred at RT under nitrogen for 30 min and sodium triacetoxyborohydride (3.05 g, 14.39 mmol) was added neat. The mixture was then stirred at 25° C. under nitrogen for 40 h, and evaporated to dryness to give an orange semi-solid. The residue was treated with ethyl acetate (200 mL), and the mixture was stirred at 40° C. for 30 min and filtered. Silica gel (2.40 g) was added to the filtrate, and the mixture was evaporated to dryness under vacuum to give silica gel impregnated with the crude product. Purification by flash column chromatography (100% ethyl acetate) gave 1.55 g (84% yield) of Compound 9 as an orange solid, mp: 66-68° C.

The free base compound 9 (0.84 g, 4 mmol) was dissolved in ethyl acetate (20 mL) and a 4N HCl solution in ethyl acetate (1.05 mL, 4.2 mmol) was added with stirring. A tan solid precipitated, and the mixture was stirred at RT for 30 min and evaporated to dryness. The residue was stirred with dry ether (80 mL) for 15 min. The ether was decanted off and discarded. This procedure (adding ether, stirring for 15 minutes and decanting off the ether) was repeated two more times. The tan solid that remained was dried under vacuum at 60° C. for 48 h to give 690 mg (70.2% yield) of the compound B10, mp: 189-190° C.

MS: 210 (MH⁺, 95), 195 (100), 139 (92).

Calc. for C₁₂H₂₂N₂ClO: C, 58.64; H, 9.02; N, 11.40; Cl, 14.42. Found: C, 58.54; H, 8.85; N, 11.07; Cl 14.01.

¹³C NMR data (δ, DMSO-d₆):

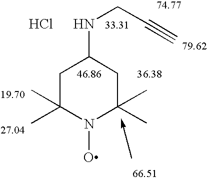

### Example 6

### 2,2,6,6-Tetramethyl-4-(methylprop-2-ynylamino)-1-piperidine nitroxide HCl (Compound B11)

Tempone (1.50 g, 8.81 mmol) was dissolved in DCE (50 mL) and a solution of N-methylpropargylamine (0.77 g, 11.07 mmol) in DCE (10 mL) was added. The mixture was stirred at RT under nitrogen for 30 min and sodium triacetoxyborohydride (3.05 g, 14.39 mmol) was added neat. The mixture was then stirred and heated at 50° C. under nitrogen for 32 h, and evaporated under reduced pressure to give an orange semi-solid. The residue was treated with ethyl acetate (200 mL), and the mixture was stirred at 40° C. for 30 min and filtered. Silica gel (2.80 g) was added to the filtrate, and the mixture was evaporated to dryness under vacuum to give silica gel impregnated with crude product. Purification by flash column chromatography (100% ethyl acetate) gave 0.70 g (36% yield) of a red oil (Compound 10).

The free base compound 10 (0.70 g, 3.13 mmol) was dissolved in ethyl acetate (20 mL) and a 4N HCl solution in ethyl acetate (0.85 mL, 3.40 mmol) was added with stirring. A tan solid precipitated and the mixture was stirred at RT for 30 min and evaporated to dryness, and the residue stirred with dry ether (60 mL) for 15 min. The ether was decanted off and discarded. This procedure (adding ether, stirring for 15 min and decanting off the ether) was repeated three more times. The tan solid that remained was dried under vacuum at 60° C. for 48 h to give 415 mg (51% yield) of compound B11, mp: 165-168° C.

MS: 224 (MH⁺, 15), 209 (5), 167 (34), 150 (54), 136 (76), 122 (100).

Calc. for C₁₃H₂₄N₂ClO: C, 60.10; H, 9.31; N, 10.79; Cl, 13.65. Found: C, 59.54; H, 9.09; N, 10.43; Cl 14.35.

### Example 7

### (2,2,6,6-Tetramethyl-1-piperidinyloxy-4-yl)-carbamic acid 3(R)-(t-butoxycarbonyl-prop-2-ynylamino-indan-5-yl ester (Compound 8)

Compound 6 was synthesized as described in U.S. Patent Publication Number US-2004-0010038-A1, published Jan. 15, 2004 (WO 2003/072055), example 4.

Bis-(trichloromethyl)carbonate (BTC, 0.345 g, 1.16 mmol) was dissolved in dioxane (20 ml), and a solution of compound 6 (1 g, 3.48 mmol) and pyridine (1.4 ml, 17.3 mmol) in dioxane (5 ml) was added slowly (within 10 min). After a few minutes of stirring at RT, 4-amino-2,2,6,6-tetramethyl piperidine-1-nitroxide (4-amino-TEMPO, 0.7 g, 4.08 mmol) in dioxane (2 ml) was added portionwise and the red mixture was stirred at RT for 3-4 h. The mixture was filtered and the filtrate evaporated to give 1.8 g of a red-brown oily residue. Flash chromatography (hexane: EtOAc-2:1) gave compound 8 (0.545 g, 32%) as a white-pinkish solid.

Anal. calcd for C₂₇H₃₉N₃O₅: C, 66.78; H, 8.09; N, 8.65, O16.47, found C, 66.70; H, 7.83; N, 8.61.

¹H NMR (CDCl₃ for two rotamers) δ: 7.20 and 6.98 (two br s, 3H, Ar), 5.80 and 5.43 (two br s, 1H, NCH), 4.16, 3.96, 3.67 and 3.42 (four br s, 2H, NCH₂), 3.00, 2.82 2.46, 2.72, 2.08 (five br s, CHCH₂CH₂ and CCH), 1.2-1.6 (br s, 9H, Boc).

MS (FAB+) 486.

### Example 8

### (2,2,6,6-Tetramethyl-1-piperidinyloxy-4-yl)-carbamic acid 3(R)-prop-2-ynylamino-indan-5-yl ester HCl (Compound A10)

Compound 8 (135 mg, 0.278 mmol) was dissolved in EtOAc (1 ml), and 4N HCl/EtOAc (2 ml, ˜8 mmol, ˜30 eq) was added in one portion at RT. After ½ h of stirring at RT, Et₂O (˜5 ml) was added to give a heterogeneous mixture which was stirred at RT for 2 h. The solid was collected by filtration and washed extensively with Et₂O to give a yellowish solid which was dried to give Compound A10 as a white solid (115 mg, 98%).

¹H NMR (DMSO d₆) δ: 10.2 (br s, 2H, N⁺H₂), 8.10 (br d, 1H, NHCO), 7.53 (br s, 1H, Ar H-7), 7.32 (br d, 1H, J=8 Hz, Ar H-4), 7.09 (br d, 1H, J=8 Hz, Ar H-5), 4.79 (br s, 1H, NHCH-1), 3.90-3.93 (br s, 2H, NHCH₂), 3.73 (br s, 1H, CCH), 3.12, 2.85, 2.46 and 2.29 (four br m, 4H, CHCH₂CH₂), 2.06-2.13 (br m, 4H, CHCH₂C(Me)₂), 1.49 and 1.34 (two s, 12H, CHCH₂C(Me)₂).

¹³C NMR (DMSO d₆) δ: 153.78 (NHCO), 149.62 (ArCO), 141.42 and 138.24 (Ar), 125.48, 123.13 and 119.28 (Ar CH), 79.56 and 75.05 (CCH), 67.10 (C(Me)₂), 60.60 (NCH-1), 41.23 (CONHCH) 41.10 (CHCH₂C(Me)₂), 33.75 (NHCH₂), 29.33 (CHCH₂CH₂), 28.47 (CHCH₂CH₂), 27.25 (Me), 20.01 (Me).

MS (FAB+) 386.

MS (CI): 384 (M⁺, 55), 331 (100), 186 (46).

### Example 9

### 4-(Indan-1-ylamino)-2,2,6,6-tetramethyl-1-piperidine nitroxide HCl (Compound C10)

Tempone (1.50 g, 8.81 mmol) was dissolved in 1,2 dichloroethane (50 ml) and a solution of R-aminoindan (1.48 g, 11.07 mmol) in dichloroethane (10 mL) was added. The mixture was stirred at RT under nitrogen for 30 min, and sodium triacetoxyborohydride (3.05 g, 14.39 mmol) was added neat. The mixture was then stirred at RT under nitrogen for 72 h, and the solvent was evaporated under reduced pressure to give an orange semi-solid. The residue was treated with ethyl acetate (200 mL), and the mixture was stirred at 35° C. for 30 min, and filtered. Silica gel (3.20 g) was added to the filtrate, and the mixture was evaporated to dryness under vacuum to give silica gel impregnated with the crude product. Purification by flash column chromatography (100% ethyl acetate) gave 1.80 g (71%) of an orange solid, compound 11, mp: 131-132° C.

The free base compound 11 (1.15 g, 4.00 mmol) was dissolved in ethyl acetate (80 mL) and a 4N HCl solution in ethyl acetate (1.05 mL, 4.2 mmol) was added with stirring. A tan solid precipitated, and the mixture was stirred at RT for 1 h, and evaporated to dryness. The residue was stirred with dry ether (100 mL) for 30 min, filtered and the collected solid was washed with dry ether (75 mL) and dried to give 640 mg (50%) of the title product, mp: 219-220° C.

Anal. calcd for C₁₈H₂₈N₂ClO: C, 66.75; H, 8.71; N, 8.65, Cl 10.95; found C, 65.89; H, 8.48; N, 8.41, Cl 10.79.

MS: 288 (MH, 34), 214 (25, MH—Me₂NHOH), 172 (27), 117 (100).

### Example 10

### 2,2,6,6-Tetramethyl-4-prop-2-ynylamino-1-piperidine-1-ol dihydrochloride (Compound B12)

To a stirred solution of compound 9 (114 mg, 0.54 mmol) in anhydrous MeOH (5 mL) a solution of L-ascorbic acid (106 mg, 0.60 mmol) in anhydrous MeOH (5 mL) was added. The mixture was stirred at RT under nitrogen for 30 min, and the solvent evaporated to dryness. The residue was impregnated onto silica gel and purified by flash column chromatography (CH₂Cl₂/MeOH, 75/25) to give 65 mg (57% yield) of 2,2,6,6-Tetramethyl-4-prop-2-ynylamino-1-piperidine-1-ol as a tan solid.

¹H NMR (CDCl₃) δ: 3.5 (d, 2H, CH₂C≡CH), 3.2 (m,1H,C4-H), 2.25 (s,1H, CH₂C≡CH), 1.85 (m,2H), 1.45 (m,2H), 1.25(d,12H).

The free base (100 mg, 0.48 mmol) was converted to the dihydrochloride salt by dissolving it in dry methanol (10 ml) and adding HCl (g) in dry ether (1 ml). The solution was stirred at RT for 30 min. and let to stand for another 30 min. The solvent was evaporated to dryness at reduced pressure to give an off-white solid, which was dried to give 100 mg of the product (74% yield).

Microanalysis: calc. for the di-HCl salt (C12H24N2 C12 O) containing 1.5 moles of water: C, 46.57; H, 8.77; N, 9.03; Cl, 22.85.

Found: C, 46.04; H, 8.34; N, 8.42; Cl, 23.27.

1H NMR (D2O): 4.06 (d, 2H, N—CH2-propargyl), 3.95(m, 1H, C4-H), 3.07 (t, 1H, CH2-propargyl-H), 2.52 (d, 2H), 2.05 (t, 2H), 1.52 s, 12H).

### BIOLOGICAL EXAMPLES

### Example 11

Evaluation of antioxidant properties of the compounds in vitro.

Hypochlorite (HOCl) Hemolysis of Human Red Blood Cells Model

The reaction is based on the ability of oxygen radicals to lyse red blood cells. Antioxidant compounds such as ascorbic acid and 4-hydroxy-TEMPO (TEMPOL) prevent membrane damage in a dose dependent manner. All compounds were dissolved at a concentration of 5 mg/ml and aliquots were tested for their ability to prevent lysis.

Chemiluminescence Model (Without Cells)

Luminescence was generated in vials containing the following compounds: Luminol, SIN-1 (a generator of NO radicals), selenite, BSA. This reaction is inhibited by scavengers of peroxynitirites as well as scavengers of NO and other oxygen free radicals.

PMA-Induced Oxidative Burst in Neutrophils Model

The cells were activated with PMA (10 ng/ml) for 30 minutes and incubated with 2,7-dichlorodihydrofluorescin diacetate (DCFH), which is converted to a fluorescent compound in the presence of oxygen radicals and peroxynitrites. The fluorescent cells were detected in a cell sorter (FACS).

Statistical Evaluation of the Results

The raw data was incorporated into Sigma-Stat and the mean±SEM of the different groups and treatments were compared using several tests suggested by the program. The results are shown in table 1.

TABLE 1

| | In vitro activity of test compounds | | |
|---|---|---|---|
| | Antioxidant activity | | |
| | | Chemi- | PMA |
| Test Compound | HOCl | luminescence | (% cells |
| (or control) | (% lysis) | (% inhib.) | activated) |
| Ascorbic acid | 100 | 100 | 100 |
| Tempol (average) | 25 | 57 | 70 |
| Compound B10 | 45 | 42 | ND |

ND = no data

### Example 12

The Parkinson Neurotoxicity Model of 1-methyl-4-phenyl piridinium (MPP+ Iodine Salt, 1000 μM for 48-52 hr), Using 7-10 Days NGF-Differentiated PC12 Cells.

The slow developing cell death in this model is believed to be a result of inhibition of mitochondrial complex I and generation of oxygen and nitrogen radicals. Therefore the antioxidant tempol (4-HYDROXY-TEMPO) was used as a positive standard in the screening experiments with the compounds. The following detailed protocol was employed for testing.

MPP+—Neurotoxicity/Neuroprotection Protocol:

1. PC12 cells (2×10⁵ cells) were seeded on NUNC 6-well dishes coated with 200 μg/ml collagen (rat tail type I) and grown in DMEM medium containing 7% Fetal Calf Serum, 7% Horse Serum and 10,000 U/ml Penicilin and 100 ug/ml Streptomycin.
2. Differentiation of the cells for 7-10 days was achieved by treatment with mouse β-NGF (β-nerve growth factor) (50 ng/ml) added freshly every 2-3 days to the culture medium.
3. Cultures were progressively evaluated for the differentiation response expressed by elongation of neurites, increased percentage of responsive cells, and other morphological parameters.
4. At the day of the experiment, the medium of the differentiated cultures was replaced to fresh medium containing NGF (50 ng/ml).
5. Thereafter, the compounds were added 45-60 min prior to the initiation of the insult with MPP+, and they were continuously present through the experiment (during the experiment the medium was not changed). A concentration of 1000 μM MPP+ was found to be optimal to induce a mild insult during 48-52 hr of exposure, therefore in all the experiments the insult was achieved at this concentration. As a positive neuroprotective control we used in all experiments 500 μM tempol. The active compounds were checked at 1 and 10 μM.
6. LDH (lactic dehydrogenase) release to the medium (neurotoxicity) was measured after 48-52 hr after the addition of MPP+, using an ELISA reader.

On the day of the experiment, compounds were dissolved in growth culture medium supplemented with NGF, or DMSO to a stock solution of 10 mM. Dilutions were made from the stock in medium containing NGF (50 ng/ml) to achieve a final concentration of either 1 or 10 μM compound and less than 0.1% DMSO.

Neurotoxicity is defined as the percentage of LDH released to the medium at the end of the experiment calculated according to the following formula:
{LDH_{(MPP)}−LDH_{(control)}}/{LDH₍ₜₒₜₐₗ₎−LDH_{(control)}}×100=Neurotoxicity (% of total cell death)
LDH_{(MPP)}: LDH release at the end of MPP+ insult
LDH_{(control)}: LDH release at the end of experiment from untreated cultures
LDH₍ₜₒₜₐₗ₎: LDH released from the cultures upon freezing at −80° C. and thawing at room temperature (release of total/maximal LDH present in the culture)

Neuroprotection is defined as the reduction in neurotoxicity reflected by the reduction in LDH release to the medium in the presence of tested compounds compared to LDH release after MPP+ insult in the absence of tested compound. Neuroprotection is calculated according to the following formula:
Neuroprotection (%)=[100−toxicity_{(with tested compound)}/toxicity_{(mpp only)}×100]

The statistical evaluation was performed by one-way analysis of variance (ANOVA). A P value of <0.05 was considered significant and was labeled by a star (*). The Dunnet Multiple Comparisons Test was performed with LDH values of the different compounds tested relative to MPP+ insult. To calculate the percentage of neuroprotection for each set of sixplicate experiments, the average neurotoxicity was calculated and the following calculation was undertaken:
100−([neurotoxicity test compound/neurotoxicity MPP⁺]×100).

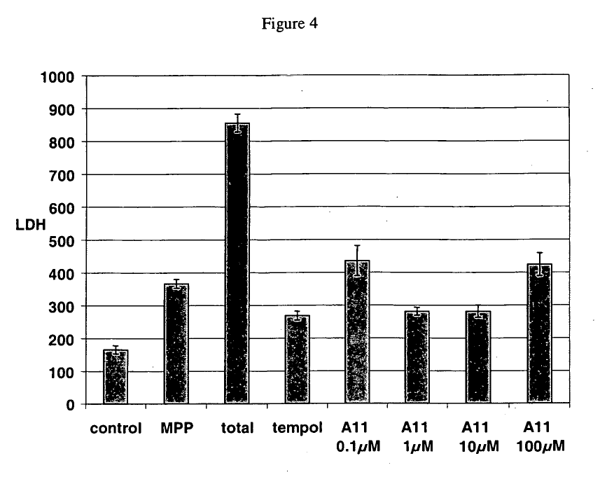

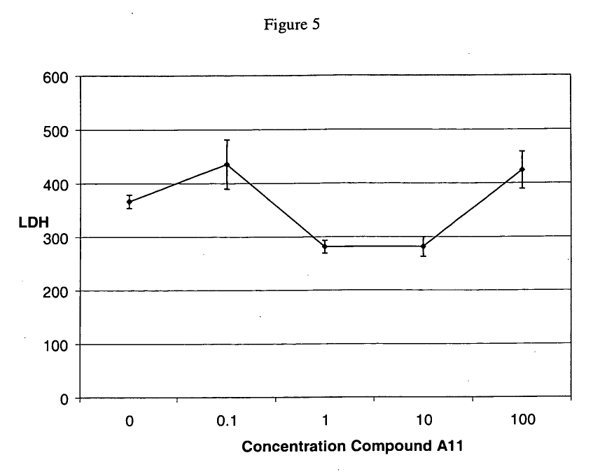

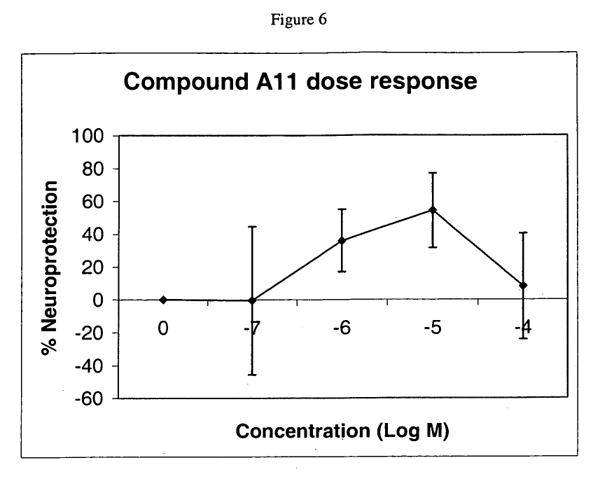

The results achieved for the compounds screened are given in FIGS. 4, 5, and 6 and in Table 2.

TABLE 2

| | In vitro activity of test compounds | |
|---|---|---|
| Test Compound | Neuroprotection | |
| (or control) | MPP+ | |
| Tempol (average) | Active at 0.5-1.0 | mmol |
| Compound B10 | Active at 1 & 10 | μM |
| Compound A11 | 43% at 10 | μM |

### Example 13

Inhibition of EAE (PLP and MOG Induced)

Induction of Experimental Allergic Encephalomyelitis (EAE) Using PLP

Female SJL mice (12 weeks old) were inoculated with the encephalitogienic peptide of proteolipid protein (PLP 139-151) synthesized to a purity of 70% by Sigma (Israel). 150 μg of the peptide were emulsified in complete Freund's adjuvant (CFA) (Difco Laboratories), supplemented with killed mycobacteria (5 mg/ml) and pertussis toxin (200 ng) (Sigma), given subcutaneously at day of inoculation only.

Mice were kept at specific pathogen free (SPF) conditions and given water and food ad libitum. Mice were daily observed for clinical signs from day 10 until day 18-21 post inoculation.

Treatment with Test Compounds

Compounds were dissolved in 2-hydroxypropyl beta cyclodextrin (40% water solution). Treatment started at day 1 post inoculation. The compounds B10 and A10 (50 mg/kg) and vehicle were given orally by gavage every day until day 20 post inoculation. The results are summarized in Table 3.

Induction of EAE Using MOG (Myelin Oligodendrocyte Glycoprotein)

Female C57B1/6 mice were inoculated (subcutaneous injection in the right flank) with the encephalitogenic emulsion (MOG plus CFA enriched with MT (mycobacterium tuberculosis)). A boost of this emulsion was injected sc in the right flank 1 week later. On the day of the first MOG injection, pertussis toxin was injected ip (0.1 ml/mouse). Test compounds were suspended in 0.5% methylcellulose and given orally by gavage. The animals were kept in SPF conditions and given food ad libitum. They were allocated to groups as shown below:

| | Compound | | | | No. of |
|---|---|---|---|---|---|
| Group | administered | dose | | administration | mice |
| 1 | Control vehicle | 0.5% | MC | Gavage ×2/day | 15 |
| 2 | A11 | 5 | mg/Kg | Gavage ×2/day | 15 |
| 3 | A11 | 25 | mg/kg | Gavage ×2/day | 15 |

Schedule of procedures during study:

| DAY | TEST PROCEDURE |
|---|---|
| 1 | Subcutaneous injection of MOG into |
| | right flank. ip injection of Pertussis toxin. |
| | Administration of drugs (gavage ×2/day) |
| 3 | ip injection of Pertussis toxin. |
| 8 | Subcutaneous injection of MOG into left flank |
| 10 | Initiation of scoring of mice for EAE clinical signs |
| 30 | Termination of drug treatment and study |

The mice were observed daily from the 10^{th} day post-EAE induction (first injection of MOG) and the EAE clinical signs were scored. The scores were recorded on observation cards according to the grades described in the table presented below.

| Score | Signs | Description |
|---|---|---|
| 0 | Normal behavior | No neurological signs. |
| 1 | Distal limp tail | The distal part of the |
| | | tail is limp and droops. |
| 2 | Complete limp | The whole tail is loose |
| | tail with righting | and droops. Animal has |
| | reflex | difficulties to return |
| | | on his feet when it is |
| | | laid on his back |
| 3 | ataxia | wobbly walk - when the |
| | | mouse walks the hind |
| | | legs are unsteady |
| 4 | early paralysis | The mouse has difficulties |
| | | standing on its hind legs |
| | | but still has remnants of |
| | | movement. |
| 5 | Full paralysis | The mouse can't move its |
| | | legs at all, it looks |
| | | thinner and emaciated. |
| | | Incontinence |
| 6 | Moribund/Death | |

Interpretation of Results:
Calculation of the Incidence of Disease

The number of sick animals in each group was summed. The percentage of sick animals in each group was calculated.

Calculation of the Mean Maximal Score (MMS)

The maximal scores of each of the 15 mice in the group were summed.

The mean maximal score of the group were calculated as follows:
Σ maximal score of each mouse/number of mice in the group.
Calculation of the Mean Disease Duration (MDD)

The mean duration of disease expressed in days was calculated as follows:
Σ duration of disease of each mouse/number of mice in the group.
Calculation of the Group Mean Score (GMS)

The scores of each of the 15 mice in the group was summed and the mean score per day was calculated.

The group mean score was calculated as follows:
Σ total score of each mouse per day/number of mice in the group.

TABLE 3
Summary of the in vivo results of the test compounds.

| | | In vivo | | | | |
|---|---|---|---|---|---|---|
| | | EAE ^{(a, b)} | | | | |
| | | Incid. | | | | |
| Model | Compound | (# dead) | MMS | MDD | MDO | Mean score |
| PLP | Control | 4/4 (0) | 5.0 ± 0 | 9.0 ± 0 | 10 ± 0 | 3.57 ± 0.29 |
| induced | Compound B10 | 5/5 (3) | 5.0 ± 0.77 | 5.2 ± 0.97 | 13.6 ± 0.81 | 3.57 ± 0.29 |
| EAE | Compound A10 | 5/5 | 5.0 ± 0.8 | 5.8 ± 0.86 | 11 ± 0.2 | 3.4 ± 0.3 |
| MOG | Control | 9/13 | 4.78 ± 0.46 | 14.67 ± 1.32 | 13.89 ± 0.48 | 2.35 ± 0.15 |
| induced | Compound A11 | 7/13 | 2.93 ± 0.76 | 8.0 ± 2.02 | 17.14 ± 0.80 | 0.65 ± 0.09 |
| EAE | | | | | | |

^{a)} MMS = mean maximal score; MDD = mean disease duration (days); MDO = mean day of onset
^{b)} dose: 50 mg/kg/day

Discussion

EAE is an accepted animal model of multiple sclerosis (see Tisch, R. And McDevitt, H. O. Proc. Natl. Acad. Sci. USA (1994) 91: 437-438 and reference cited therein.) As such, the results above suggest that the compounds of the present invention would be effective for treating MS in humans.

In addition, oxidative stress has been implicated in a variety of neurologic diseases, as discussed in the background of the invention. (See, for example, M. P. Mattson et al., J. Neurosci. Res. (1997) 49:681). As illustrated by the examples above, the compounds of the present invention are effective antioxidants and free radical scavengers. This data, evaluated in light of the EAE experimental data presented above, suggests that the compounds of the present invention would be effective treatments for a variety of neurologic diseases which involve oxidative stress.

Furthermore, the results indicate that Compound A11 showed a significant beneficial effect on EAE. In particular, treatment with Compound A11 resulted in a long delay in the onset of clinical signs of EAE. This delay may indicate that the compounds have an effect on cell activation or proliferation. Consequently, Compound A11 may be effective for treating neurologic diseases which have an effect on cell activation or cell proliferation.

1. A compound having the structure: 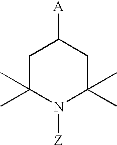
wherein Z is —OH or —O•; and
A is: 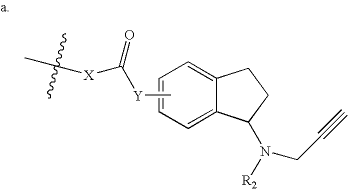
wherein
X and Y are independently NR₁ or O, where
R₁ is H or C₁-C₄ alkyl; and
R₂ is H, C₁-C₄ alkyl or t-butoxycarbonyl, 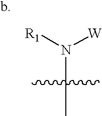
wherein W is C₃-C₄ alkynyl; and
R₁ is H or C₁-C₄ alkyl, or 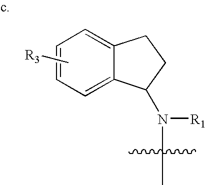
wherein R₁ is H, C₁-C₄ alkyl, or C₃-C₄ alkynyl; and
R₃ is H, OH, O(C₁-C₄ alkyl), or a halogen,
or an enantiomer or a pharmaceutically acceptable salt thereof.

2. The compound of claim 1, having the structure: 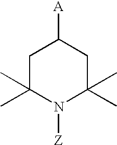
wherein
Z is —OH or —O•; and
A is: 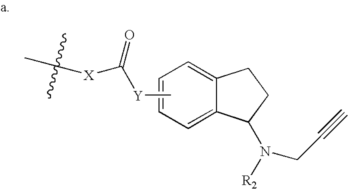
wherein
X and Y are independently NR₁ or O, where
R₁ is H or C₁-C₄ alkyl; and
R₂ is H, C₁-C₄ alkyl, 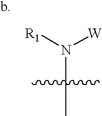
wherein W is C₃-C₄ alkynyl; and
R₁ is H or C₁-C₄ alkyl, or 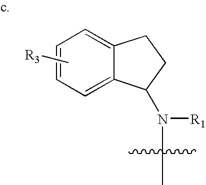
wherein R₁ is H, C₁-C₄ alkyl, or C₃-C₄ alkynyl;
and R₃ is H, OH, O(C₁-C₄ alkyl), or a halogen,
or an enantiomer or a pharmaceutically acceptable salt thereof.

3. The compound of claim 2, wherein A is: 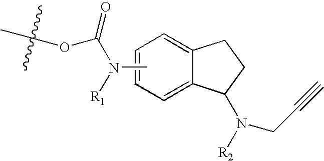
wherein R₁ is H or C₁-C₄ alkyl; and
R₂ is H or C₁-C₄ alkyl,
or an enantiomer or a pharmaceutically acceptable salt thereof.

4. The compound of claim 3, wherein R₁ and R₂ are H.

5. The compound of claim 2, wherein Z is —O•.

6. The compound of claim 5, wherein the compound is (3-prop-2-ynylamino-indan-5-yl) carbamic acid 2,2,6,6-tetramethyl-1-piperidinyloxy-4-yl ester HCl.

7. The compound of claim 2 wherein A is: 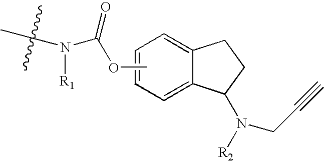
wherein
R₁ is H or C₁-C₄ alkyl; and
R₂ is H or C₁-C₄ alkyl,
or an enantiomer or a pharmaceutically acceptable salt thereof.

8. The compound of claim 7, wherein R₁ and R₂ are H.

9. The compound of claim 7, wherein Z is —O•.

10. The compound of claim 9, wherein the compound is (2,2,6,6-tetramethyl-1-piperidinyloxy-4-yl)-carbamic acid 3-(R)-prop-2-ynylamino-indan-5-yl ester HCl.

11. The compound of claim 2, wherein A is 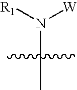
wherein W is C₃-C₄ alkynyl; and
R₁ is H or C₁-C₄ alkyl,
or an enantiomer or a pharmaceutically acceptable salt thereof.

12. The compound of claim 11, wherein A is

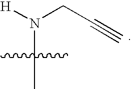

13. The compound of claim 11, wherein Z is —O•.

14. The compound of claim 13, wherein the compound is 2,2,6,6-tetramethyl-4-prop-2-ynylamino-1-piperidine nitroxide HCl.

15. The compound claim 2, wherein Z is —OH.

16. The compound of claim 15, wherein the compound is 2,2,6,6-tetramethyl-4-prop-2-ynylamino-piperidin-1-ol 2HCl.

17. The compound of claim 11, wherein A is

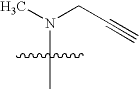

18. The compound of claim 17, wherein Z is —O•.

19. The compound of claim 18, wherein the compound is 2,2,6,6-tetramethyl-4-(methyl-prop-2-ynylamino)-1-piperidine nitroxide HCl.

20. The compound of claim 2, wherein A is 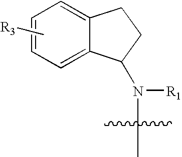
wherein R₁ is H, C₁-C₄ alkyl, or C₃-C₄ alkynyl; and
R₃ is H, OH, O(C₁-C₄ alkyl), or a halogen,
or an enantiomer or a pharmaceutically acceptable salt thereof.

21. The compound of claim 2, wherein R₁ and R₃ are H.

22. The compound of claim 2, wherein Z is —O•.

23. The compound of claim 22 wherein the compound is 4-(indan-1-ylamino)-2,2,6,6-tetramethyl-1-piperidine nitroxide HCl.

24. The compound of claim 1, wherein the compound is an optically active enantiomer.

25. The compound of claim 1, having the structure: 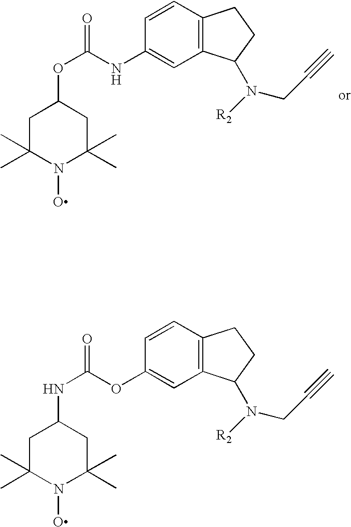
wherein R₂ is a t-butoxycarbonyl group.

26. A process of manufacturing the compound of claim 5 comprising:
a. reacting 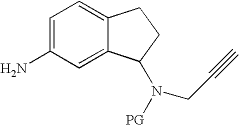
wherein PG is a protecting group, with (CCl₃O)₂CO to form: 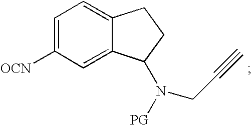
b. reacting the product of a. with 4-hydroxy-2,2,6,6-tetramethylpiperidine-1-nitroxide to form: 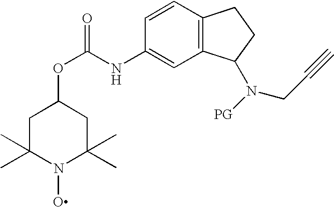
wherein PG is a protecting group; and
c. reacting the product of step b. with an acid to form:

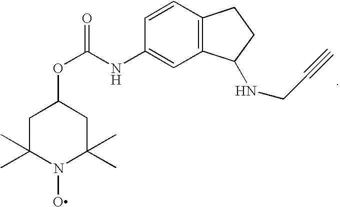

27-28. (canceled)

29. A process of manufacturing a compound of claim 9 comprising:
a. reacting 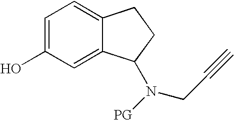
wherein PG is a protecting group, with bis-(trichloromethyl) carbonate to form: 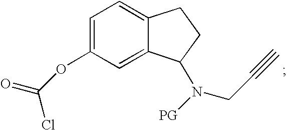
b. reacting the product of step a. with 4-amino-2,2,6,6-tetramethyl piperidine-1-nitroxide to form: 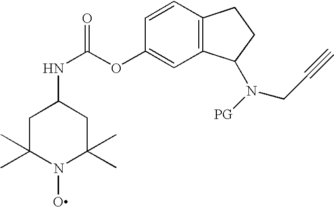
wherein PG is a protecting group; and
c. reacting the product of step b. with an acid to form:

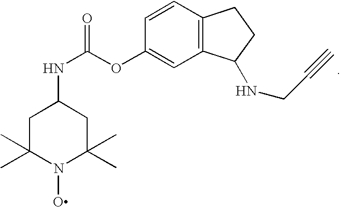

30. (canceled)

31. A process of manufacturing a compound having the structure: 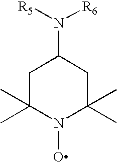
wherein R₅ is C₃-C₄ alkynyl or an indan-1-yl group and
R₆ is H or C₁-C₄ alkyl, comprising:
a. reacting a compound having the structure: 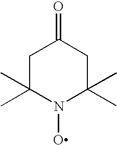
with a compound having the structure: 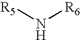
wherein R₅ and R₆ are defined as above, in the presence of a reducing agent to form the compound.

32-34. (canceled)

35. A method of treating a subject suffering from a neurologic disorder or an autoimmune disorder, comprising administering to the subject a therapeutically effective amount of the compound of claim 2 so as to thereby treat the subject.

36-41. (canceled)

42. A method of treating a subject afflicted with an inflammatory disorder caused by the presence of reactive oxygen species, comprising administering to the subject a therapeutically effective amount of the compound of claim 2 so as to thereby treat the subject.

43-46. (canceled)

47. A method of preventing the oxidation of lipids, proteins, or deoxyribonucleic acid in a cell, comprising contacting the cell with the compound of claim 2.

48. A method of preventing lysis of human red blood cells by oxygen radicals, comprising contacting the cells with the compound of claim 2.

49. A pharmaceutical composition comprising the compound of claim 2 and a pharmaceutically acceptable carrier.

50. A process for the manufacture of a pharmaceutical composition comprising admixing the compound of claim 2 with a pharmaceutically acceptable carrier.

51-66. (canceled)
